(19) United States
(12) Patent Application Publication
(10) Pub. No.: US 2012/0203714 A1
(43) Pub. Date: Aug. 9, 2012

(54) Systems for Growing Phototrophic Organisms Using Green Energy

(75) Inventors: Jaime A. Gonzalez, Oakville (CA); Max Kolesnik, Schomberg (CA); Steven C. Martin, Toronto (CA)

(73) Assignee: POND BIOFUELS INC., Scarborough (CA)

(21) Appl. No.: 13/021,489

(22) Filed: Feb. 4, 2011

(51) Int. Cl. G06Q 90/00; C12N 1/00; C12N 5/04; C12M 1/00; C12M 3/00

(52) U.S. Cl. 705/500; 435/289.1; 435/292.1; 435/419; 435/243

(57) ABSTRACT

A system for producing phototrophic biomass is provided. The system includes a green power generator and a carbon sequestering system. The green power generator is provided for generating power, wherein operation of the green power generator is controlled by an environmentally friendly business organization. The carbon sequestering system is electrically coupled to the green power generator for receiving at least a fraction of the power generated by the green power generator for powering a phototrophic biomass production process that is operational within the carbon sequestering system. Operation of the carbon sequestering system is controlled by the same environmentally friendly business organization. A method of producing phototrophic biomass is also provided. The method includes generating power with a green power generator, wherein operation of the green power generator is controlled by an environmentally friendly business organization.

### TECHNICAL FIELD

The present disclosure relates generally to reducing carbon footprint.

### BACKGROUND

In typical phototrophic biomass production systems, a light source is powered by electricity in order to grow the phototrophic biomass. Since electricity is supplied by local power companies, phototrophic biomass producers have little, if any, control over the source of the electricity being used to grow the phototrophic biomass. Local power companies often use greenhouse gas-producing fuels such as coal and natural gas for generating electricity, which releases large amounts of carbon dioxide into the air along with other toxins. The otherwise green phototrophic biomass production process is contaminated in a way that significantly increases the carbon footprint of an organization engaged in the phototrophic biomass production process, which is particularly undesirable for such organizations, which typically strive to be as green as possible.

### SUMMARY

One aspect of the present disclosure provides a system for producing phototrophic biomass. The system includes a green power generator and a carbon sequestering system. The green power generator is provided for generating power, wherein operation of the green power generator is controlled by an environmentally friendly business organization. The carbon sequestering system is electrically coupled to the green power generator for receiving at least a fraction of the power generated by the green power generator for powering a phototrophic biomass production process that is operational within the carbon sequestering system. Operation of the carbon sequestering system is controlled by the same environmentally friendly business organization.

In another aspect, a method of producing phototrophic biomass is provided. The method includes generating power with a green power generator, wherein operation of the green power generator is controlled by an environmentally friendly business organization. The generated power is supplied to power a phototrophic biomass production process that is operational within a carbon sequestering system, and thereby effect production of the phototrophic biomass. Operation of the carbon sequestering system is controlled by the same environmentally friendly business organization.

In another aspect, there is provided a use of an operative biofuel. The operative biofuel is either: (a) phototrophic biomass produced in accordance with a method comprising: generating power with a green power generator, wherein operation of the green power generator is controlled by an environmentally friendly business organization, and supplying the generated power to power a phototrophic biomass production process that is operational within a carbon sequestering system, and thereby effect production of the phototrophic biomass, wherein operation of the carbon sequestering system is controlled by the same environmentally friendly business organization, or (b) a phototrophic biomass derivation product that is produced by processing the produced phototrophic biomass.

In another aspect, there is provided a system for producing phototrophic biomass. The system includes a solar energy utilization system and a carbon sequestering system. The solar energy utilization system includes a solar collector system, a light energy supply system, and a solar energy conversion and converted energy supply system. The carbon sequestering system, in which a phototrophic biomass production process is operational, includes a reaction zone configured for containing a reaction mixture that is operative for effecting photosynthesis upon exposure to photosynthetically active light radiation. The solar collector system is configured for receiving incident solar radiation and interacting with the received incident solar radiation to effect production of a light source-purpose received incident solar radiation fraction and an energy source-purpose received incident solar radiation fraction. The light energy supply system is configured for supplying photosynthetically active light radiation to the reaction zone and thereby effecting exposure of the reaction mixture to the photosynthetically active light radiation. The solar energy conversion and converted energy supply system is configured for converting the energy source-purpose received incident solar radiation fraction to converted energy and supplying the converted energy to the phototrophic biomass production process.

### BRIEF DESCRIPTION OF THE DRAWINGS

Reference will now be made to the drawings, which show by way of example, embodiments of the present disclosure, and in which:

FIG. 1 shows a block diagram for a system for effecting production of a phototrophic biomass using green energy;

FIG. 2 shows a block diagram for a system for effecting use of phototrophic biomass, or a phototrophic biomass-derived product, in generating power for power consumers;

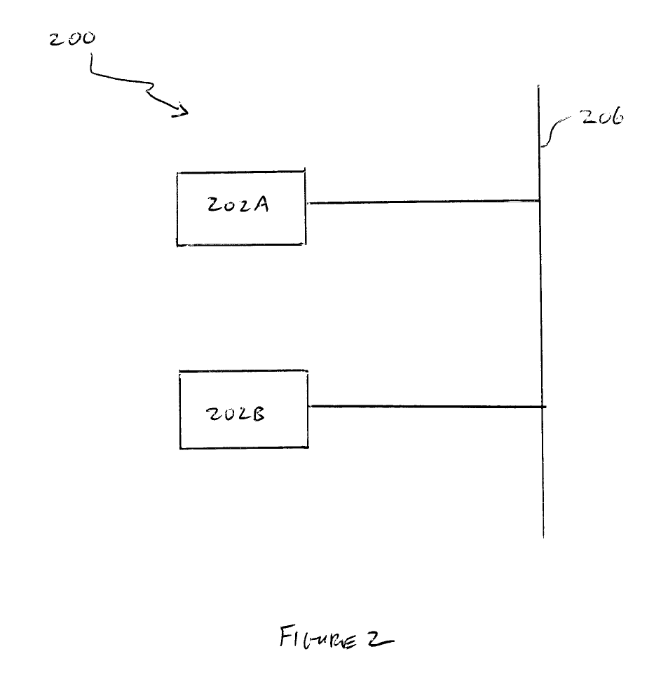

FIG. 3 shows a block diagram for a system for producing green energy for effecting production of phototrophic biomass and also for supplying to power consumers;

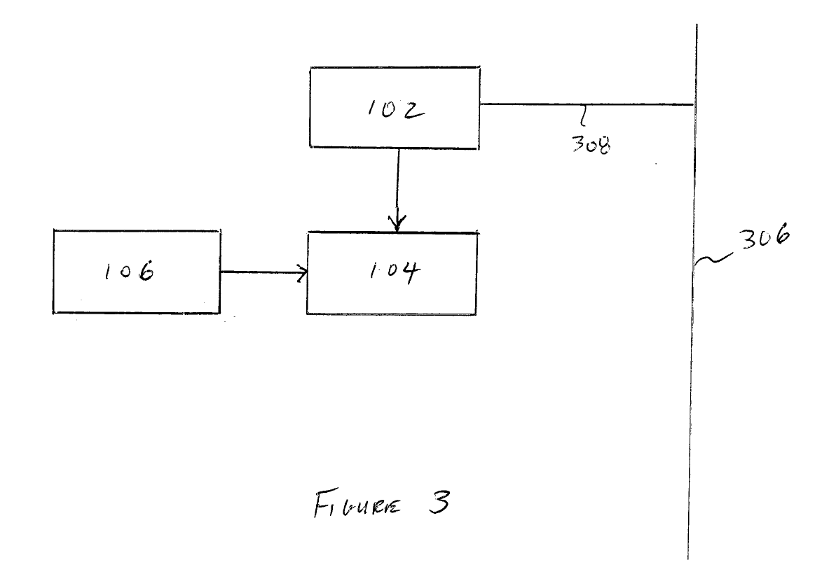

FIG. 4 shows a block diagram for a system for generating green energy for effecting growth of phototrophic biomass across a distance;

FIG. 5 is a schematic illustration of an embodiment of a system for producing phototrophic biomass; and

FIG. 6 is a schematic illustration of another embodiment of a system for producing phototrophic biomass.

### DETAILED DESCRIPTION

Reference throughout the specification to “some embodiments” means that a particular feature, structure, or characteristic described in connection with some embodiments are not necessarily referring to the same embodiments. Furthermore, the particular features, structure, or characteristics may be combined in any suitable manner with one another.

There is provided a green (or, “environmentally friendly”) power generator 102.

Examples of suitable sources of energy for the green power generator 102 include nuclear energy, wind energy, geothermal energy, solar energy, biomass, biofuels, and hydraulic energy. Examples of suitable green power generators include solar power generators, wind power generators, nuclear power generators, and hydro-electric power generators.

In some embodiments, for example, the green power generator 102 is a power generator that effects discharge, into the atmosphere, of less than 0.7 pounds of carbon dioxide per kilowatt hour of power generated. In some embodiments, for example, the green power generator 102 is a power generator that effects generation of power, and at least 50% of the generated power is effected from a renewable energy source. Examples of suitable renewable energy sources include wind energy, solar energy, geothermal energy, biomass, biofuels and hydraulic energy.

The green power generator 102 is electrically coupled to a carbon sequestering system 104 for powering one or more electrical loads included within the carbon sequestering system 104.

The carbon sequestering system 104 is a system that includes a phototrophic biomass production process that is configured to receive a carbon-comprising gas and effect removal (or capture) and sequestration of at least a fraction of the carbon from the received carbon-comprising gas.

A phototrophic biomass production process is operational in the carbon sequestering system 104. The phototrophic biomass production process effects the growing of a phototrophic biomass in a reaction zone 10. The reaction zone 10 includes a reaction mixture that is operative for effecting photosynthesis upon exposure to photosynthetically active light radiation. The reaction mixture includes phototrophic biomass material, carbon dioxide, and water. In some embodiments, the reaction zone includes phototrophic biomass and carbon dioxide disposed in an aqueous medium. Within the reaction zone, the phototrophic biomass is disposed in mass transfer communication with both of carbon dioxide and water. The carbon comprising-gas is received within the reaction zone for supplying the reaction mixture with carbon dioxide. The phototrophic biomass production process includes exposing the reaction mixture disposed in the reaction zone to photosynthetically active light radiation so as to effect photosynthesis. The photosynthesis effects growth and, therefore, production of phototrophic biomass. In some embodiments, the reactive process also effects production of a phototrophic biomass production process gaseous discharge. Any phototrophic biomass production process gaseous discharge is at least depleted in carbon relative to the carbon-comprising gas. In some embodiments, for example, any phototrophic biomass production process gaseous discharge includes substantially no carbon.

“Phototrophic organism” is an organism capable of phototrophic growth in the aqueous medium upon receiving light energy, such as plant cells and micro-organisms. The phototrophic organism is unicellular or multicellular. In some embodiments, for example, the phototrophic organism is an organism which has been modified artificially or by gene manipulation. In some embodiments, for example, the phototrophic organism is algae. In some embodiments, for example, the algae is microalgae.

“Phototrophic biomass” is at least one phototrophic organism. In some embodiments, for example, the phototrophic biomass includes more than one species of phototrophic organisms.

“Reaction zone 10” defines a space within which the growing of the phototrophic biomass is effected. In some embodiments, for example, the reaction zone is provided in a photobioreactor 12.

“Photobioreactor 12” is any structure, arrangement, land formation or area that provides a suitable environment for the growth of phototrophic biomass. Examples of specific structures which can be used is a photobioreactor by providing space for growth of phototrophic biomass using light energy include, without limitation, tanks, ponds, troughs, ditches, pools, pipes, tubes, canals, and channels. Such photobioreactors may be either open, closed, partially closed, covered, or partially covered. In some embodiments, for example, the photobioreactor is a pond, and the pond is open, in which case the pond is susceptible to uncontrolled receiving of materials and light energy from the immediate environments. In other embodiments, for example, the photobioreactor is a covered pond or a partially covered pond, in which case the receiving of materials from the immediate environment is at least partially interfered with. The photobioreactor includes the reaction zone which includes the reaction mixture. In some embodiments, the photobioreactor is configured to receive a supply of phototrophic reagents (and, in some of these embodiments, optionally, supplemental nutrients), and is also configured to effect discharge of phototrophic biomass which is grown within the reaction zone. In this respect, in some embodiments, the photobioreactor includes one or more inlets for receiving the supply of phototrophic reagents and supplemental nutrients, and also includes one or more outlets for effecting the recovery or harvesting of biomass which is grown within the reaction zone. In some embodiments, for example, one or more of the inlets are configured to be temporarily sealed for periodic or intermittent time intervals. In some embodiments, for example, one or more of the outlets are configured to be temporarily sealed or substantially sealed for periodic or intermittent time intervals. The photobioreactor is configured to contain the reaction mixture which is operative for effecting photosynthesis upon exposure to photosynthetically active light radiation. The photobioreactor is also configured so as to establish photosynthetically active light radiation (for example, a light of a wavelength between about 400-700 nm, which can be emitted by the sun or another light source) within the photobioreactor for exposing the phototrophic biomass. The exposing of the reaction mixture to the photosynthetically active light radiation effects photosynthesis and growth of the phototrophic biomass. In some embodiments, for example, the established light radiation is provided by an artificial light source disposed within the photobioreactor. For example, suitable artificial lights sources include submersible fiber optics or light guides, light-emitting diodes (“LEDs”), LED strips and fluorescent lights. Any LED strips known in the art can be adapted for use in the photobioreactor. In the case of the submersible LEDs, in some embodiments, for example, energy sources include alternative energy sources, such as wind, photovoltaic cells, fuel cells, etc. to supply electricity to the LEDs. Fluorescent lights, external or internal to the photobioreactor, can be used as a back-up system. In some embodiments, for example, the established light is derived from a natural light source which has been transmitted from externally of the photobioreactor and through a transmission component. In some embodiments, for example, the transmission component is a portion of a containment structure of the photobioreactor which is at least partially transparent to the photosynthetically active light radiation, and which is configured to provide for transmission of such light to the reaction zone for receiving by the phototrophic biomass. In some embodiments, for example, natural light is received by a solar collector, filtered with selective wavelength filters, and then transmitted to the reaction zone with fiber optic material or with a light guide. In some embodiments, for example, both natural and artificial lights sources are provided for effecting establishment of the photosynthetically active light radiation within the photobioreactor.

“Aqueous medium” is an environment that includes water. In some embodiments, for example, the aqueous medium also includes sufficient nutrients to facilitate viability and growth of the phototrophic biomass. In some embodiments, for example, supplemental nutrients may be included such as one of, or both of, NOₓ and SOₓ. Suitable aqueous media are discussed in detail in: Rogers, L. J. and Gallon J. R. “Biochemistry of the Algae and Cyanobacteria,” Clarendon Press Oxford, 1988; Burlew, John S. “Algal Culture: From Laboratory to Pilot Plant.” Carnegie Institution of Washington Publication 600. Washington, D.C., 1961 (hereinafter “Burlew 1961”); and Round, F. E. The Biology of the Algae. St Martin's Press, New York, 1965; each of which is incorporated herein by reference). A suitable supplemental nutrient composition, known as “Bold's Basal Medium”, is described in Bold, H. C. 1949, The morphology of Chlamydomonas chlamydogama sp. nov. Bull. Torrey Bot. Club. 76: 101-8 (see also Bischoff, H. W. and Bold, H. C. 1963, Phycological Studies IV. Some soil algae from Enchanted Rock and related algal species, Univ. Texas Publ. 6318: 1-95, and Stein, J. (ED.) Handbook of Phycological Methods, Culture methods and growth measurements, Cambridge University Press, pp. 7-24).

The phototrophic biomass production process, when operational, consumes at least a fraction of the power being generated and supplied by the green power generator 102 to the carbon sequestering system 104. In some embodiments, for example, the electrical load provided in the carbon sequestering system for the phototrophic biomass production process and being powered by the green power generator is an artificial light source configured for supplying photosynthetically active light radiation to the reaction zone 10 and thereby effecting exposure of the reaction mixture to the photosynthetically active light radiation. With respect to artificial light sources, for example, suitable artificial light sources include submersible fibre optics, light-emitting diodes (“LEDs”, including submersible LEDs), LED strips, and fluorescent lights. In some embodiments, for example, the electrical load provided in the phototrophic biomass production process and being powered by the green power generator is any one of valves, sensors, controllers, blowers, fans, dampers, and pumps. For example, the electrical load is a pump for supplying make-up water to the reaction zone 10 and/or for supplying supplemental nutrients to the reaction zone. For example, the electrical load is a prime mover (such as a blower or a fan) for supplying the carbon comprising gas to the reaction zone. For example, the electrical load is a dryer for effecting drying of phototrophic biomass-comprising product recovered from the reaction zone.

In some embodiments, for example, the green power generator 102 includes a solar energy utilization system 1000. The solar energy utilization system 1000 includes a solar collector system 1100, a light energy supply system 1200, and a solar energy conversion and converted energy supply system 1300.

The solar collector system 1100 is configured for receiving incident solar radiation and interacting with the received incident solar radiation to effect production of a light source-purpose received incident solar radiation fraction and an energy source-purpose received incident solar radiation fraction. In some embodiments, for example, the interacting includes separating the light source-purpose received incident solar radiation fraction and the power generation-purpose received incident solar radiation fraction from the received incident solar radiation. The light source-purpose received incident solar radiation fraction is supplied to the light energy supply system 1200. The light source-purpose received incident solar radiation fraction includes photosynthetically active light radiation. The energy source-purpose received incident solar radiation fraction is supplied to the solar energy conversion and converted energy supply system 1300.

The light energy supply system 1200 is configured for supplying photosynthetically active light radiation of the light source-purpose received incident solar radiation fraction to the reaction zone 10 provided in the carbon sequestering system 104 and thereby effecting exposure of the reaction mixture to the photosynthetically active light radiation. The solar energy conversion and converted energy supply system 1300 is configured for converting the energy source-purpose received incident solar radiation fraction to converted energy and supplying the converted energy to the phototrophic biomass production process.

In some embodiments, for example, the solar energy conversion and converted energy supply system 1300 is configured for converting the energy source-purpose received incident solar radiation fraction to electricity and supplying the electricity for powering an electrical load provided in the carbon sequestering system 104 and thereby supplying energy to the phototrophic biomass production process. In this respect, in some embodiments, for example, the solar energy conversion and converted energy supply system 1300 includes a photovoltaic cell 1002 for effecting the conversion of the energy source-purpose received incident solar radiation fraction to electricity.

Alternatively, in some embodiments, the energy conversion and converted energy supply system 1300 is configured for converting at least a fraction of the received incident solar radiation to thermal energy and supplying the thermal energy to one or more heat sinks provided in the phototrophic biomass production process. In this respect, in some embodiments, for example, the energy conversion and converted energy supply system 1300 includes a solar heater for absorbing the incident solar radiation and converting the received incident solar radiation into thermal energy of a working fluid of the solar heater. The heated working fluid is then supplied to various heat sinks provided in the phototrophic biomass production process, such as dryer 206 for effecting drying of phototrophic biomass-comprising product recovered from the reaction zone 10.

In some embodiments, for example, the solar collector system 1100 includes a filter/mirror assembly 1006 (including, for example, any one of an interference filter/mirror assembly, a dielectric elliptical mirror, or a dichromic mirror filter) that filters the received incident solar radiation to provide a light source-purpose received incident solar radiation fraction and a power generation-purpose received incident solar radiation fraction. The light source-purpose received incident solar radiation fraction is of desirable wavelengths for purposes of effecting photosynthesis upon its exposure to the reaction mixture within the reaction zone 10. In some embodiments, for example, the light source-purpose received incident solar radiation fraction is of a light of a wavelength between about 400-700 nm. In some embodiments, for example, the light source-purpose received incident solar radiation fraction is visible light. The light source-purpose received incident solar radiation fraction is transmitted to the reaction zone with a light transmission component 1012 of the light energy supply system 1200. The light transmission component 1012 includes one or more of a waveguide, light guide, liquid light guide, light tube, or optical fibre. The power generation-purpose received incident solar radiation fraction is transmitted to a photovoltaic cell 1002 of the solar energy conversion and converted energy supply system 1300, for conversion to electricity for powering one or more of the electrical loads.

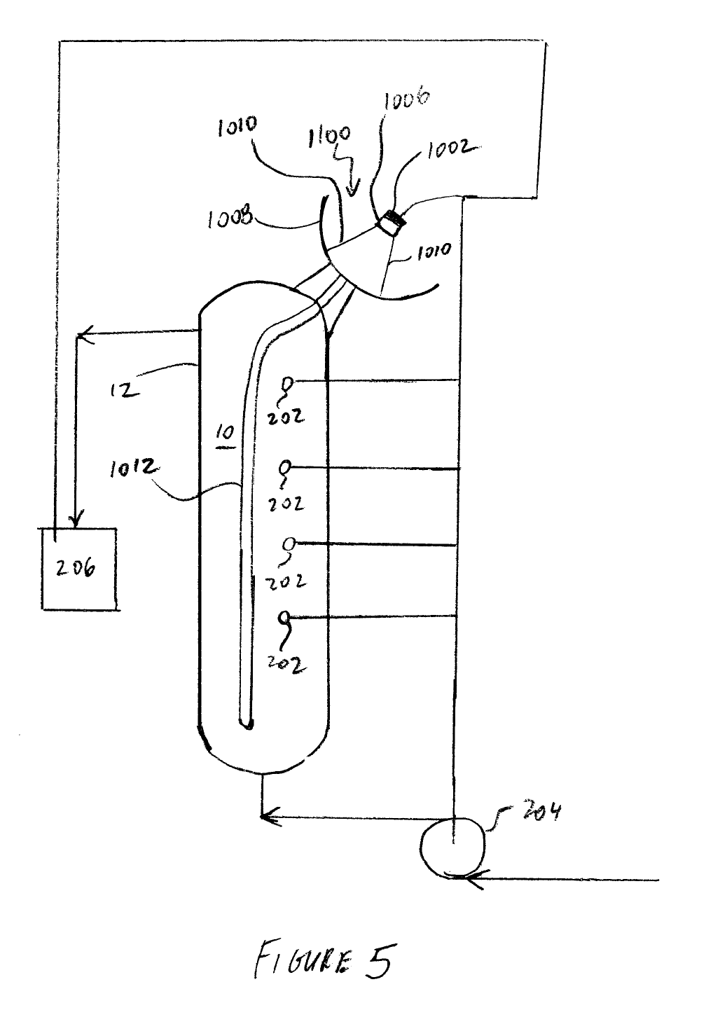

Referring to FIG. 5, in some embodiments, for example, the solar collector system 1100 includes a reflective, parabolic dish 1008 and the interference filter/mirror assembly 1006. The interference filter is mounted to and supported by the dish 1008 with supports 1010. The photovoltaic cell 1002 is mounted behind the interference filter/mirror assembly 1006 for receiving the power generation-purpose received incident solar radiation fraction. The parabolic dish 1008 is configured to reflect (with parabolic focus) incident solar radiation that impinges on the dish 1008 onto the interference filter/mirror assembly 1006. In some embodiments, for example, the interference filter/mirror assembly 1006 is a cold mirror which reflects visible light while transmitting infrared light. The interference filter/mirror assembly 1006 is configured to reflect, focus, and concentrate the light source-purpose received incident solar radiation fraction of the incident solar radiation (reflected by the dish 1008) onto a light transmission component 1012, which supplies the received light source-purpose received incident solar radiation fraction to the reaction zone 10. The interference filter/mirror assembly 1006 is also configured to permit the transmission of the power generation-purpose received incident solar radiation fraction through to the photovoltaic cell 1002. The photovoltaic cell 1002 converts the received power generation-purpose received incident solar radiation fraction to electricity for powering one or more of the electrical loads of the carbon sequestering system 104 that are used to supply energy to the phototrophic biomass production process which is operational in the carbon sequestering system 104. For example, the one or more electrical loads include LED lighting 202. For example, the one or more of the electrical loads include a prime mover 204 (such as a blower or a fan) for supplying the carbon comprising gas to the reaction zone. For example, the one or more of the electrical loads include the dryer 206 for effecting drying of phototrophic biomass-comprising product recovered from the reaction zone 10.

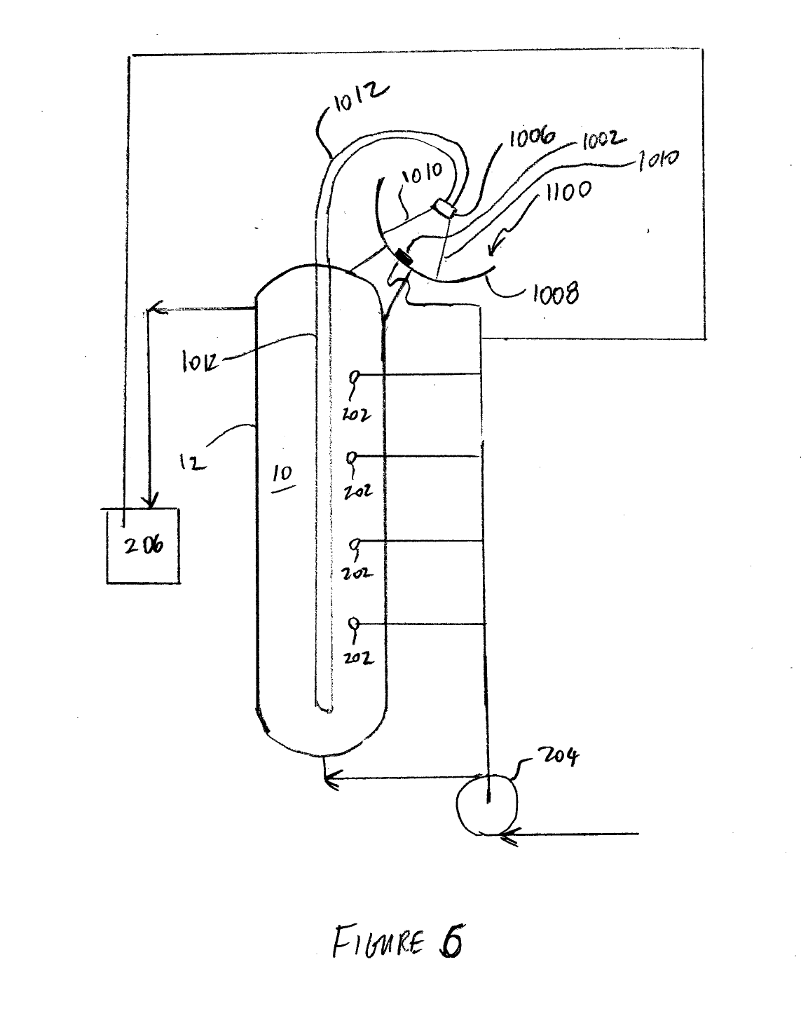

Another configuration of the solar collector 1004 is illustrated in FIG. 6. In this configuration, the light transmission component extends from behind the interference filter/mirror assembly 1006 and the photovoltaic cell 1002 is positioned relative to the dish 1008 to receive focussed and concentrated power generation-purpose received incident solar radiation fraction that is being reflected from the interference filter/mirror assembly 1006.

The operation of the green power generator 102 and the operation of carbon sequestering system 104 are controlled by the same environmentally friendly business organization. In the context of this patent application, a business organization consists of one or more participating organizations. A participating organization takes the form of any one of a corporation, a partnership, or a sole proprietorship. In this respect, the business organization consists of one or more corporations, one or more sole proprietorships, or one or more partnerships, or any combination of the foregoing. For example, a business organization may consist of one corporation, one partnership, and one sole proprietorship. In some embodiments, for example, control of the operation of a first system (for example, the power generator 102) and the control of operation of a second system (the carbon sequestering system 104) may be considered to be by the same business organization even though a first business organization (for example, consisting of Green Electricity Corp. and Sunshine Inc.) controls operation of the first system (for example, the power generator 102) and a second business organization (for example, Stopcarbon Inc.) controls operation of the second system (for example, the carbon sequestering system 104), wherein the first business organizations consists of different participating organization (in this example, Green Electricity Corp. and Sunshine Inc.) than the second business organization (in this example, Stopcarbon Inc.). This would be the case if the first and second business organizations are commonly owned or controlled (this would be the case if the shares of each of Green Electricity Corp., Sunshine Inc., and Stopcarbon Inc. were wholly owned by Planetsaver Corp.), or one of the first and the second business organizations is owned or controlled by the other (for example, this would be the case if Stopcarbon Inc. owns all of the shares of each of Green Electricity Corp. and Sunshine Inc.). So long as there is commonality in control of the decision making power of the business organization controlling operation of the first system (for example, the power generator 102) and in control of the decision making power of the business organization controlling operation of the second system (for example, the carbon sequestering system 104), for the purposes of this patent application, the control of operation of the first system (for example, the power generator 102) and the control of operation of the second system (for example, the carbon sequestering system 104) would be considered to be by the same business organization. Conversely, for purposes of this patent application, when such commonality in control is lacking as between business organizations with different participating organizations, such business organizations are considered to be different.

In some embodiments, a carbon-comprising exhaust gas discharging system 106 is also provided and is configured to effect production of a carbon-comprising exhaust gas. The carbon-comprising exhaust gas discharging system 106 is fluidly coupled to the phototrophic biomass production process. A carbon-comprising exhaust gas discharging process is operational at the carbon-comprising exhaust gas discharging system 106. At least a fraction of the carbon-comprising exhaust gas discharge is supplied to the phototrophic biomass production process as the carbon-comprising gas. In some embodiments, for example, the carbon-comprising exhaust gas discharging system 106 is co-located with the carbon sequestration system 104 on the same property.

The carbon-comprising exhaust gas discharging process includes any process which effects production of the carbon-comprising exhaust gas. In some embodiments, for example, the carbon-comprising exhaust gas discharging process is a combustion process being effected in a combustion facility. In some of these embodiments, for example, the combustion process effects combustion of a carbon-comprising material. A suitable carbon-comprising material is a fossil fuel, such as coal, oil, or natural gas. For example, the combustion facility is any one of a fossil fuel-fired power plant, an industrial incineration facility, an industrial furnace, an industrial heater, or an internal combustion engine. In some embodiments, for example, the combustion facility is a cement kiln.

In some embodiments, for example, the environmentally friendly business organization receives a quantified credit for the power being generated by the green power generator 102, such as a carbon offset or a carbon credit. In some embodiments, for example, the quantified credit is provided on the basis that discharge of at least some carbon dioxide into the atmosphere is mitigated or eliminated by generating green power with the green power generator and using the generated green power in the carbon sequestering process, relative to using power generated from other than a green power generator.

In some embodiments, for example, the environmentally friendly business organization receives a quantified credit for effecting sequestration of carbon dioxide by the carbon sequestering system 104, and the quantified credit is, for example, a carbon offset or a carbon credit. In some embodiments, for example, the quantified credit is provided on the basis that discharge of at least some carbon dioxide into the atmosphere is mitigated or eliminated.

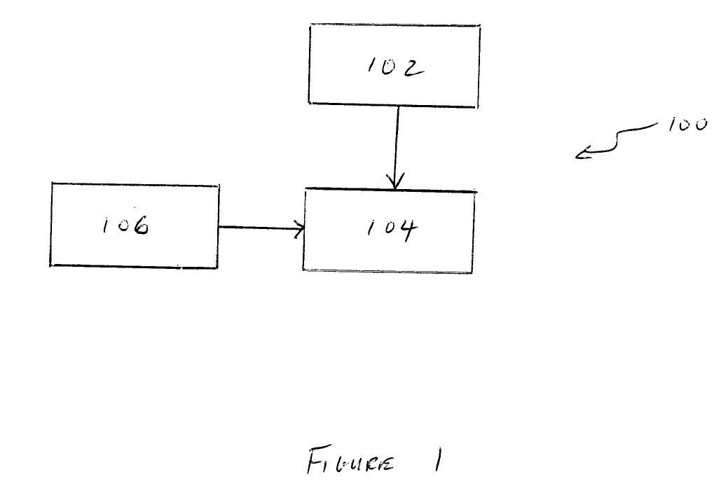

Referring now to FIG. 1, a block diagram is shown illustrating an embodiment of a system 100. The system 100 includes the green power generator 102 electrically coupled to the carbon sequestering system 104.

In another aspect, there is provided a method of producing phototrophic biomass using any of the embodiments of the system 100 described above. In this respect, power is generated by the green power generator 102. At least a fraction of the power generated by the green power generator 102 is received by the carbon sequestering system 104 for powering one or more processes that are operational in the carbon sequestering system 104, the one or more processes including the phototrophic biomass production process. The phototrophic biomass production process is operational within the carbon sequestering system. The operation of the power generator 102 and the operation of the carbon sequestering system 104 are controlled by the same environmentally friendly business organization. The received power is used by the carbon sequestering system 104 to power the phototrophic biomass production process and thereby effect production of phototrophic biomass by the phototrophic biomass production process. In some embodiments, for example, the green power generator includes the solar energy utilization system 1000, as described above, for powering the carbon sequestering system 104 and also for supplying photosynthetically active light radiation to the reaction zone 10 for effecting exposure of the reaction mixture to the photosynthetically active light radiation and thereby effecting production of the phototrophic biomass.

In another aspect, an operative biofuel is provided, wherein the operative biofuel is either the produced phototropic biomass or is a phototrophic biomass-derived upgraded biofuel. The phototrophic biomass-derived upgraded biofuel is derived from the produced phototrophic biomass by converting the produced phototrophic biomass to the phototrophic biomass-derived upgraded biofuel, wherein the phototrophic biomass-derived upgraded biofuel is characterized by a higher energy density relative to the produced phototrophic biomass. In some embodiments, for example, production of the phototrophic biomass-derived upgraded biofuel is effected as follows. The produced phototrophic biomass is gasified to effect production of an intermediate syngas, and subjecting the intermediate syngas to the Fishcer-Tropsch reactive process to effect production of the phototrophic biomass-derived upgraded biofuel.

In this respect, use of the operative biofuel is also provided, and the use includes combustion of the operative biofuel to effect production of energy. In some embodiments, for example, the operative biofuel is combusted in an industrial process for effecting production of energy that is consumed within the industrial process, such as in the making of cement, steel, etc. In some embodiments, for example, the operative biofuel is combusted in an internal combustion engine.

In some embodiments, for example, the green power generator 102 includes a biofuel-based power generator, and at least a fraction of the power supplied to the carbon sequestering system is generated by the combustion of at least a fraction of the operative biofuel by the biofuel-based power generator, and the operation of the green power generator 102 and the operation of carbon sequestering system 104 are controlled by the same environmentally friendly business organization. In some embodiments, for example, about 20 to 30% of the fuel derived from the phototrophic biomass produced by the phototrophic biomass production process of the carbon sequestering system is provided to effect generation of power by the biofuel-based generator of the green power generator 102, with the remaining phototrophic biomass available for other uses, including processing to produce the operative biofuel for other purposes.

In some embodiments, at least a fraction of the operative biofuel is used by a business organization that is a green power generating business organization. In this respect, a system 200 is provided including a biofuel-based power generator 202A and non-biofuel-based generator 202B, wherein operation of the biofuel-based power generator 202A and operation of the non-biofuel-based power generator 202B are controlled by the same green power generating business organization. The biofuel-based power generator 202A is configured to effect generation of power by combusting any received operative biofuel. The non-biofuel-based power generator 202B is configured to effect generation of power by conversion of an energy source other than the operative biofuel into the generated power. Examples of suitable non-biofuel-based power generators 202A include solar power generators, wind power generators, nuclear power generators, and hydro-electric power generators. Each of the biofuel-based power generator 202A and the non-biofuel-based power generator 202B is electrically coupled to a power transmission system 206 for providing respectively generated power to the power transmission system 206. The power transmission system 206 is configured to effect transmission of the provided power to power consumers. In this respect, there is provided a method of generating green power by effecting combustion of the operative biofuel in a biofuel-based power generator 202A, wherein operation of the biofuel-based power generator 202A is controlled by a green power generating business organization, and wherein operation of a non-biofuel-based power generator 202B is also controlled by the same green power business organization. The generated green power is supplied to power consumers through the power transmission system 206. In some embodiments, for example, the green power generating business organization is different than the environmental friendly business organization.

In some embodiments, the generation of power by the biofuel-based power generator 202A is effected when the energy source for the non-biofuel-based power generator 202B is unavailable for effecting power generation by the non-biofuel-based power generator 202B. For example, when the non-biofuel-based power generator 202B is a wind power generator, power generation by the biofuel-based power generator is effected when insufficient wind is available for powering the wind power generator. As a further example, when the non-biofuel-based power generator 202B is a solar power generator, power generation by the biofuel-based power generator is effected when insufficient sunlight (for example, during periods of darkness, such as during the night) is available for powering the solar power generator.

In another aspect, at least for some period of operation of the green power generator 102, such operation being controlled by the environmentally friendly business organization, at least a fraction of the green power that is generated by the green power generator 102 is supplied to power consumers. In this respect, there is provided a system 300 including the green power generator 102, the carbon sequestration system 104, and a power transmission system 306. The power transmission system 306 is electrically coupled to the green power generator 102 with a transmission line 308. The power transmission system 306 effects transmission of the generated and supplied green power to power consumers. In some embodiments, for example, while the green power generator 102 is effecting generation of the green power, a fraction of the generated green power is supplied to the power transmission system 306, and a fraction of the generated green power is supplied to the carbon sequestering system 104 (whose operation is also controlled by the same environmentally friendly organization). In some embodiments, for example, while the green power generator 102 is effecting generation of the green power, and a fraction of the generated green power is being supplied to the power transmission system 306, when demand for electricity by power consumers decreases, supply of the generated green power to the carbon sequestering system 104 is either initiated or increased.

In some embodiments, for example, the environmentally friendly business organization receives a quantified credit for the using of the operative biofuel (whether by the environmentally friendly business organization itself, or by a different business organization), including the using of the operative biofuel as above-described. In some embodiments, for example, the quantified credit is provided on the basis that use of the operative biofuel (such as by combustion of the operative biofuel) mitigates or eliminates the use of a non-renewable source of energy (for example, combustion of coal or other fossil fuel) for the generation of energy (for example, heat or electricity).

In some embodiments, for example, any of the above-described quantified credits is usable by the environmentally friendly business organization to obtain a tax credit. In some embodiments, for example, any of the above-described quantified credits is usable by the environmentally friendly business organization to offset a carbon tax applicable to operation of the carbon sequestering system 104, or to offset a carbon tax applicable to operation of the carbon-comprising exhaust gas discharging system 106 which is supplying the carbon-comprising gas to the phototrophic biomass production process. In some embodiments, for example, any of the above-described quantified credits is transferable to a different business organization so as to enable the different business organization to obtain a tax credit or to offset any carbon tax that is applicable to the different business organization.

In some embodiments, for example, any of the above-described received quantified credits offset at least a fraction of the carbon tax applicable to those embodiments of the carbon sequestering system 104 which receive some non-green power (non-green power refers to power that is generated by other than a green power generator) to effect operation of processes within the carbon sequestering system 104 (including that used to operate the phototrophic biomass production process). In some embodiments, for example, any of the above-described received quantified credits offset at least a fraction of carbon being output from an other than a green power generator 102 that is supplying a fraction of the power being supplied to the carbon sequestering system 104 to effect operation of processes within the carbon sequestering system 104 (including that used to operate the phototrophic biomass production process). In some embodiments, for example, any of the above-described received quantified credits is useable by the environmentally friendly business organization to offset a carbon tax applicable to any carbon-comprising exhaust gas discharging process that is exhausting carbon dioxide-comprising exhaust gas, a fraction of which is being supplied to the phototrophic biomass production process, and a fraction of which is being discharged to the atmosphere. In some embodiments, for example, any of the above-described received quantified credits is useable by the environmentally friendly business organization to offset a carbon output of any carbon-comprising exhaust gas discharging system 106 that is exhausting carbon dioxide-comprising exhaust gas, a fraction of which is being supplied to the phototrophic biomass production process, and a fraction of which is being discharged to the atmosphere.

The examples above are intended to be merely exemplary and the quantified credit is intended to include any currently available credit or any credit available in the future that may potentially provide favourable treatment for the environmentally friendly business organization, or for another business organization that is transferred such quantified credit either directly or indirectly from the environmentally business organization.

In another aspect, there is provided a system 400 including the green power generator 102 and the carbon sequestering system 104, wherein the operation of the green power generator 102 and the operation of carbon sequestering system are controlled by the same environmentally friendly business organization, where the green power generator 102 is not provided at the same location as a carbon sequestering system 104.

The green power is generated at a location that is remote from the carbon sequestering system 104. In some embodiments, for example, this is effected because land use constraints limit the opportunity to supply power to the carbon sequestering system 104 at the same location that hosts the carbon sequestering system 104.

In this respect, in one aspect, the green power generator 102 is separated from the carbon sequestering system 104 by a closest separation distance of at least one (1) kilometre. In some embodiments, for example, the green power generator 102 is separated from the carbon sequestering system 104 by a closest separation distance of at least five (5) kilometres. In some embodiments, for example, the green power generator 102 is separated from the carbon sequestering system 104 by a closest separation distance of at least ten (10) kilometres. In some embodiments, for example, the green power generator 102 is separated from the carbon sequestering system 104 by a closest separation distance of at least 100 kilometres.

In another aspect, an impassable real property is disposed between the green power generator 102 and the carbon sequestering system 104. In this context, “impassable real property” means that the environmentally friendly business organization does not enjoy a right of way to effect transmission of power generated by the green power generator 102 to the carbon sequestering facility 104. In some embodiments, for example, the impassable real property is owned by a legal entity other than any legal entity included within the environmentally friendly business organization.

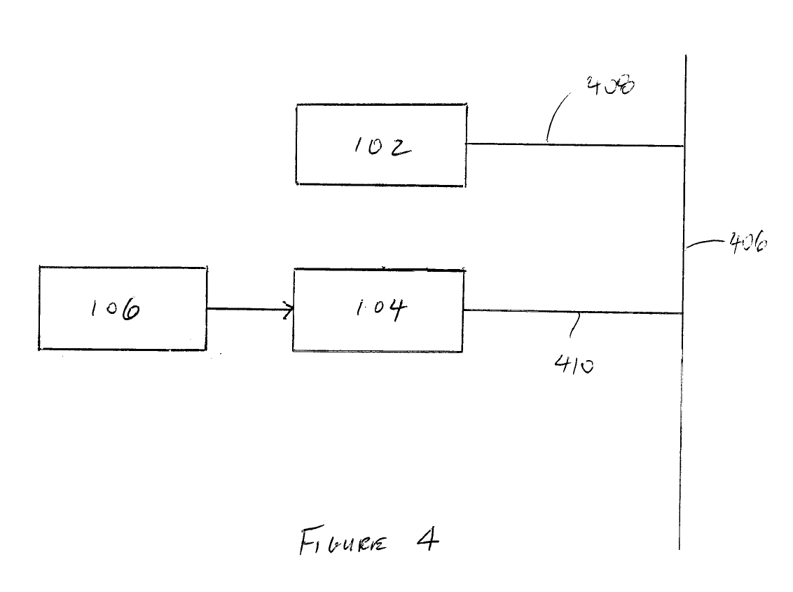

Referring now to FIG. 4, a block diagram is shown illustrating an embodiment of the system 400. The system 400 including the power generator 102 electrically coupled to a power transmission system 406 by a transmission line 408. The system 400 further comprises the carbon sequestering system 104 electrically coupled to a transmission line 410 that electrically couples the carbon sequestering system to the power transmission system 406.

The system 400 includes the green power generator 102. As described above, the green power generator 102 uses an green power source for generating power. The green power generator 102 supplies the generated power to the power transmission system 406, for example, through the transmission line 408. The power transmission system 406 is electrically coupled to the power generator 102 for receiving the power generated by the power generator 102 and supplying the power to power consumers. The carbon sequestering system 104 is electrically coupled to the transmission system 406, for example through a transmission line 410, for receiving power from the transmission system 406 for powering the phototrophic biomass production process that is operational in the carbon sequestering system 104.

Any of the power transmission systems 206, 306, 406 described above may include any suitable transmission lines, transformers, power grids, substations, and any other components known to those skilled in the relevant arts. Any of the power transmission systems 206, 306, 406 described above may span part of a city or most of a continent, depending on the design criteria of a particular application. The operation of any of the power transmission systems 206, 306, 406 described above is controlled by an organization that is unrelated to the business organization that controls the operation of at least one of the green power generator 102 and the carbon sequestering facility system 104. Any of the power transmission systems 206, 306, 406 described above is typically either state-owned or operated, or owned or operated by one or more publicly owned or privately owned electricity utility companies, but this is not always necessarily the case.

The embodiments of the present disclosure described above are intended to be examples only. Those of skill in the art may effect alterations, modifications and variations to the particular embodiments without departing from the intended scope of the present disclosure. In particular, selected features from one or more of the above-described embodiments may be combined to create alternative embodiments not explicitly described, features suitable for such combinations being readily apparent to persons skilled in the art. The subject matter described herein in the recited claims intends to cover and embrace all suitable changes in technology.

What is claimed is:

1. A system for producing phototrophic biomass, comprising:
a green power generator for generating power, wherein operation of the green power generator is controlled by an environmentally friendly business organization; and
a carbon sequestering system electrically coupled to the green power generator for receiving at least a fraction of the power generated by the green power generator for powering a phototrophic biomass production process that is operational within the carbon sequestering system, wherein operation of the carbon sequestering system is controlled by the same environmentally friendly business organization.

2. The system of claim 1, wherein
the green power generator is configured for supplying the generated power to power consumers through a power transmission system, the power transmission system being electrically coupled to the power generator for receiving the power generated by the green power generator and supplying the power to the power consumers;
the carbon sequestering system is electrically coupled to the transmission system for effecting the receiving of at least a fraction of the power generated by the green power generator for use by the phototrophic biomass production process; and
the power generator and the carbon sequestering facility are physically separated by a closest spacing distance of at least one kilometre.

3. The system of claim 1, further comprising a carbon-comprising exhaust gas discharging process that produces a carbon-comprising exhaust gas, wherein at least a fraction of the carbon-comprising exhaust gas discharge is configured to be supplied to the phototrophic biomass production process.

4. The system of claim 3, wherein the carbon-comprising exhaust gas discharging process is a combustion process.

5. The system of claim 1, wherein the green power generator includes at least one of a nuclear power generator, a solar power generator, a wind power generator, and a hydro-electric generator.

6. The system of claim 1, wherein the environmentally friendly business organization receives a quantified credit based on the generated power supplied to the carbon sequestering system.

7. The system of claim 1, wherein the environmentally friendly business organization receives a quantified credit based on the carbon dioxide that is sequestered by the phototrophic biomass production process.

8. The system of claim 1, wherein
the green power generator is configured for supplying the generated power to power consumers through a power transmission system, the power transmission system being electrically coupled to the power generator for receiving the power generated by the green power generator and supplying the power to the power consumers;
the carbon sequestering system is electrically coupled to the transmission system for effecting the receiving of at least a fraction of the power generated by the green power generator for use by the phototrophic biomass production process; and
an impassable real property is disposed between the green power generator and the carbon sequestering system.

9. The system of claim 8, wherein the environmentally friendly business organization enjoys no right of way across the impassable real property.

10. The system of claim 8, wherein the impassable real property is owned by a legal entity other than any legal entity included within the environmentally friendly business organization.

11. The system of claim 8, wherein the power generator and the carbon sequestering facility are physically separated by a closest spacing distance of at least one kilometre.

12. A method of producing phototrophic biomass, comprising:
generating power with a green power generator, wherein operation of the green power generator is controlled by an environmentally friendly business organization; and
supplying the generated power to power a phototrophic biomass production process that is operational within a carbon sequestering system, and thereby effect production of the phototrophic biomass, wherein operation of the carbon sequestering system is controlled by the same environmentally friendly business organization.

13. The method of claim 12, wherein the environmentally friendly business organization receives a quantified credit based on the generated power supplied to the carbon sequestering system.

14. The method of claim 13, wherein the environmentally friendly business organization receives a further quantified credit based on the carbon dioxide that is sequestered by the phototrophic biomass production process.

15. The method of claim 14, further comprising supplying a carbon-comprising gas to the phototrophic biomass production process to effect production of the phototrophic biomass through photosynthesis.

16. Use of an operative biofuel, wherein the operative biofuel is either:
(a) phototrophic biomass produced in accordance with a method comprising:
generating power with a green power generator, wherein operation of the green power generator is controlled by an environmentally friendly business organization; and
supplying the generated power to power a phototrophic biomass production process that is operational within a carbon sequestering system, and thereby effect production of the phototrophic biomass, wherein operation of the carbon sequestering system is controlled by the same environmentally friendly business organization;
or
(b) a phototrophic biomass-derived upgraded biofuel, wherein production of the phototrophic biomass-derived biofuel is effected by conversion of the produced phototrophic biomass.

17. The use of claim 16, wherein the use includes combusting the operative biofuel.

18. The method of claim 17, wherein the environmentally friendly business organization receives a quantified credit based on the operative biofuel which is combusted.

19. The method of claim 18, wherein the environmentally friendly business organization receives a further quantified credit based on the green power supplied to the carbon sequestering system.

20. The method of claim 19, wherein the environmentally friendly business organization receives a further quantified credit based on the carbon dioxide that is sequestered by the phototrophic biomass production process.

21. A system for producing phototrophic biomass, comprising:
a solar energy utilization system including a solar collector system, a light energy supply system, and a solar energy conversion and converted energy supply system;
a carbon sequestering system in which a phototrophic biomass production process is operational, including a reaction zone configured for containing a reaction mixture that is operative for effecting photosynthesis upon exposure to photosynthetically active light radiation;
wherein the solar collector system is configured for receiving incident solar radiation and interacting with the received incident solar radiation to effect production of a light source-purpose received incident solar radiation fraction and an energy source-purpose received incident solar radiation fraction;
wherein the light energy supply system is configured for supplying photosynthetically active light radiation to the reaction zone and thereby effecting exposure of the reaction mixture to the photosynthetically active light radiation; and
wherein the solar energy conversion and converted energy supply system is configured for converting the energy source-purpose received incident solar radiation fraction to converted energy and supplying the converted energy to the phototrophic biomass production process.

22. The system of claim 21, wherein the interacting with the received incident solar radiation includes separating the light source-purpose received incident solar radiation fraction and the power generation-purpose received incident solar radiation fraction from the received incident solar radiation.
